(19) United States
(12) Patent Application Publication
(10) Pub. No.: US 2004/0254645 A1
(43) Pub. Date: Dec. 16, 2004

(54) Prosthetic patellar implant

(76) Inventors: Uri Arnin, Kiryat Tivon (IL); Yossi Tuval, Netanya (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 10/865,910

(22) Filed: Jun. 14, 2004

Related U.S. Application Data
(60) Provisional application No. 60/478,354, filed on Jun. 16, 2003.

(51) Int. Cl. A61F 2/38

(52) U.S. Cl. 623/20.2

(57) ABSTRACT

An article including a prosthetic patellar implant attachable to a posterior portion of a patella and including an elastomeric cushioning element adapted for articulation with articular surfaces posterior to the patella, the elastomeric cushioning element including an inner elastomeric layer and an outer elastomeric layer.

### CROSS-REFERENCE TO RELATED APPLICATIONS

This application claims priority under 35 USC §119 to U.S. Provisional Patent Application Ser. No. 60/478,354, filed on Jun. 16, 2003, which is incorporated herein by reference.

### FIELD OF THE INVENTION

The present invention relates to human prostheses, and more particularly to prosthetic patellar implant.

### BACKGROUND OF THE INVENTION

The knee joint is generally regarded (for example, as described in Gray's Anatomy) as a joint that consists of three articulations: two condyloid joints, one between each condyle of the femur and the corresponding meniscus and condyle of the tibia; and a third between the patella and the femur, partly arthrodial, but not completely so, since the articular surfaces are not mutually adapted to each other, so that the movement is not a simple gliding one. In simplistic terms, the patella articulates, among other things, with trochlear surfaces between the patella and the femur.

A portion or all of a knee joint may become arthritic or deformed. The surgical process for treating knee disorders with an implant is known as Total Knee Arthroplasty (TKA) or Partial Knee Arthroplasty (PKA). In TKA the bone and cartilage at the distal end of the femur and at the proximal end of the tibia are removed and replaced with plastic, metal, and/or ceramic parts. Frequently, the articulating surfaces of the patella and sulcus (opposing inner surface) may also be replaced. Because of the tendons connected to the patella, it is generally advisable to replace only the articulating surface. A less drastic procedure, the Partial Knee Arthroplasty (PKA) can be performed where the damage to the cartilage is restricted to one confined area of the knee. In the event that the osteoarthritis is confined strictly to the patellofemoral joint, a PKA is performed whereby the underside of the patella may be replaced with a patellar implant and the counter surface receives a metal trochlear groove implant. In case of isolated anterior knee pain wherein the native sulcus is not diseased or damaged, it may be useful to replace only the patella's cartilage with a patella implant.

The most common patellar implant is usually fashioned from a hard plastic (ultra high molecular weight polyethylene) in the form of a button, with or without a metal base element. This implant replaces the interior side of the patella, adjacent the femoral condyles. To implant such a prosthesis, the posterior surface of the patella is resected to produce a flat surface on which the prosthesis is mounted using cement. The patellar implant is configured to slide up and down either the natural trochlear groove (when it was not replaced in a PKA) or an artificial trochlear groove of the femoral component, which is metal or ceramic.

There are several drawbacks involved with the use of a button fashioned from hard plastic. The button is configured to replace the functions of the natural patella as it moves against the trochlear groove. However because of the nature of the anatomical shape of the groove whether natural or artificial, there are usually only a few points of contact between the button and the groove. These contact points (usually only two points) vary as the patella moves up and down the groove. The contact stresses are high which may cause damage to the natural groove or may wear away the button as it is constantly tracking up and down the metal groove in case of an artificial counter surface.

Many methods have been used in the prior art for attaching a patella implant to the native patella underside. A typical patellar implant includes pegs (usually three) on its rear surface, which must be perfectly aligned before attachment. This is a difficult task requiring surgical precision to ensure a perfect alignment so that the patella implant tracks up and down against the counter surface groove without diverting tangentially. Maltracking may occur for example when the implant is in the correct location on patella but the pegs are not (e.g., instead of being positioned at “noon-four o'clock-eight o'clock”, the pegs are at “one o'clock-five o'clock-nine o'clock), thus causing a grooved patella implant to misalign. This patellar misalignmentand resulting abnormal tracking may produce significant shearing forces and excessive contact stress that may cause degeneration of the natural articular cartilage or of the patellar implant when articulating against the metal groove in case of replacement of the natural groove. Additionally or alternately, the pegs may be aligned with the correct orientation but they are shifted slightly to the right or left so that the implant's dome area is not aligned with the target recess of the artificial or natural groove.

### SUMMARY OF THE INVENTION

The present invention is directed to a prosthetic implant, which can be used to provide resilience to the articulating anterior of a patella, as is described more in detail hereinbelow.

There is thus provided in accordance with an embodiment of the present invention an article including a prosthetic patellar implant attachable to a posterior portion of a patella and including an elastomeric cushioning element adapted for articulation with articular surfaces posterior to the patella, the elastomeric cushioning element including an inner elastomeric layer and an outer elastomeric layer.

The prosthetic patellar implant can include one or more of the following features. For example, the elastomeric cushioning element may be attached to a base element which is more rigid than the elastomeric cushioning element, the base element being attachable to the posterior portion of the patella. The base element may include fasteners protruding therefrom which are fastenable to the posterior portion of the patella. The outer elastomeric layer may include an outer convex articulating surface. The inner layer may be more resilient than the outer layer. The outer layer may be harder than the inner layer. The inner elastomeric layer may be attached to an annular ridge protruding from the base element.

### BRIEF DESCRIPTION OF THE DRAWINGS

In the drawings:

FIG. 1 is a sagittal section of a right knee joint;

FIG. 2 is a side cross-sectional view of a prosthetic patellar implant, constructed and operative in accordance with an embodiment of the present invention; and

FIG. 3 is a side cross-sectional view of the prosthetic patellar implant, in accordance with an embodiment of the present invention; and

FIG. 4 is a sagittal section of a right knee joint, including a prosthetic patellar implant constructed and operative in accordance with another embodiment of the present invention.

### DESCRIPTION OF EMBODIMENTS

Reference is now made to FIG. 1, which illustrates a typical knee joint. The patella 101 is located in front of the lower part of the femur 102. The quadriceps femoris 103 extends from the patella 101 on the superior side thereof and connects to the femur 102, and the ligamentum patellae 104 extends from the patella 101 on the inferior side thereof and connects to the tibia 105. The bursa 106 is interposed between the quadriceps femoris 103 and the front of the femur 102.

According to Gray's Anatomy, the articular surface of the patella is indistinctly divided into seven facets—upper, middle, and lower horizontal pairs, and a medial perpendicular facet. When the knee is forcibly flexed, the medial perpendicular facet is in contact with the semilunar surface on the lateral part of the medial condyle of the femur. The semilunar surface is a prolongation backward of the medial part of the patellar surface. As the leg is carried from the flexed to the extended position, first the highest pair, then the middle pair, and lastly the lowest pair of horizontal facets is successively brought into contact with the patellar surface of the femur. In the extended position, when the quadriceps femoris is relaxed, the patella lies loosely on the front of the lower end of the femur.

It is noted that the present invention is not limited to the correctness or accuracy of the above description of the articulation of the patella with the femur. Rather, the invention is valid for any theory of articulation of the patella with the femur, and provides articulation surfaces that articulate with any corresponding portion of the knee joint.

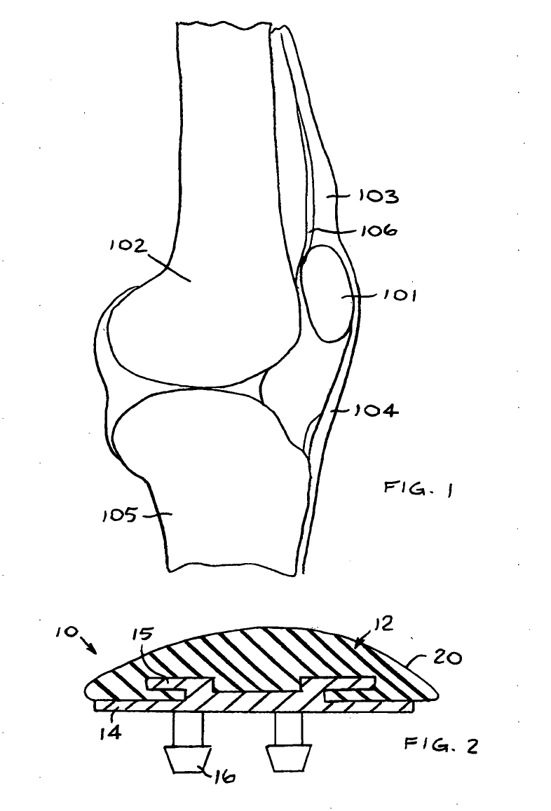

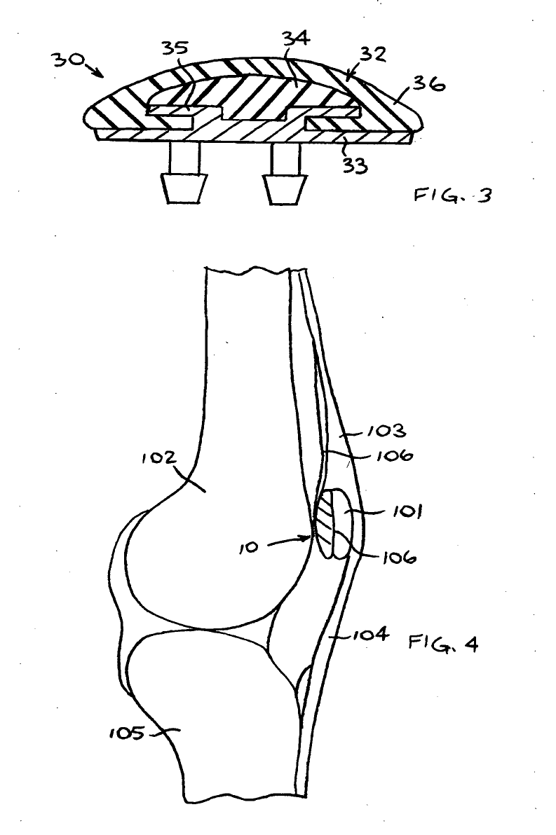

Reference is now made to FIGS. 2 and 3, which illustrate a prosthetic patellar implant 10, constructed and operative in accordance with embodiments of the present invention.

The prosthetic patellar implant 10 may be attachable to a posterior portion 106 (FIG. 4) of the patella 101. The prosthetic patellar implant 10 may include an elastomeric cushioning element 12 adapted for articulation with articular surfaces posterior to the patella 101, such as but not limited to, the condyles. The elastomeric cushioning element 12 may be constructed of an elastomeric material, such as but not limited to, polyurethane or polyurethane containing materials, silicone or silicone containing materials, polyethylene based elastomers, hydrogels, and polypropylene containing materials, and the like, or any combination thereof.

The elastomeric cushioning element 12 may be attached to a base element 14, such as by fitting over an annular ridge 15 protruding from base element 14. Base element 14 may be more rigid than elastomeric cushioning element 14, and may be constructed of a sturdy material, such as but not limited to, stainless steel, titanium alloy, cobalt chromium alloys, ceramics, or other hard, rigid materials, or different plastics, such as but not limited to, nylon, DELRIN or polyurethane, for example. The base element 14 may be attached to the posterior portion of the patella 101, such as, without limitation, by means of one or more fasteners 16 protruding therefrom. Fasteners 16 may include pegs or screws or other suitable hardware, for example.

In accordance with an embodiment of the present invention, elastomeric cushioning element 12 includes an outer convex articulating surface 20. Surface 20 is preferably resilient (compliant, the terms being used interchangeably) and its resiliency may help it to conform to the contours of the opposing articulating surfaces of the knee joint, e.g., the trochlear groove, whether natural or not. The compliance of surface 20 enables the prosthetic patellar implant 10 to articulately contact the knee joint with much a greater contact area than the prior art, thereby achieving a better stress distribution which more closely mimics the natural patellar cartilage. During movement of the leg, the patella moves in relationship to the opposing articulating surfaces of the knee joint, e.g., the trochlear groove. The compliant articulating surface 20 may alter its geometry as it moves and its contour may dynamically change to match the opposing articulating surface. This dynamic contour change may compensate for at least some of the abovementioned patellar misalignment and resulting abnormal tracking.

Reference is now made to FIG. 3, which illustrates a prosthetic patellar implant 30, constructed and operative in accordance with another embodiment of the present invention. In this embodiment, implant 30 may include an elastomeric cushioning element 32 that includes an inner elastomeric layer 34 and an outer elastomeric layer 36. The inner layer 34 may be more resilient than the outer layer 36. Outer layer 36 may be harder than the inner layer 34. For example, both inner and outer layers 34 and 36 may be constructed of polyurethane, except that the inner layer 34 has a higher Shore hardness than the outer layer 36. As another example, outer layer 36 may be constructed of Ultra-High-Molecular-Weight-Polyethylene (UHMWPe) and inner layer 34 may be constructed of polyurethane. The outer layer 36 may be more rigid or harder since it is the surface that is in constant articulating contact with the surfaces of the knee joint. The inner layer 34 may be attached to an annular ridge 35 protruding from a base element 33 (constructed similarly to that of FIG. 2). The embodiment of FIG. 3 with its two elastomeric layers 34 and 36 may have enhanced resilience and articulation properties, and ability to change shape.

Although various specific implementations have been described, it is evident that many alternatives, modifications and variations will be apparent to those skilled in the art. Accordingly, other alternatives, modifications, and variations fall within the scope of the following claims.

What is claimed is:

1. An article comprising:
a prosthetic patellar implant attachable to a posterior portion of a patella and comprising an elastomeric cushioning element adapted for articulation with articular surfaces posterior to the patella, said elastomeric cushioning element comprising an inner elastomeric layer and an outer elastomeric layer.

2. The article according to claim 1, wherein said elastomeric cushioning element is attached to a base element which is more rigid than said elastomeric cushioning element, said base element being attachable to the posterior portion of the patella.

3. The article according to claim 2, wherein said base element comprises fasteners protruding therefrom which are fastenable to the posterior portion of the patella.

4. The article according to claim 2, wherein said outer elastomeric layer comprises an outer convex articulating surface.

5. The article according to claim 1, wherein said inner elastomeric layer is more resilient than said outer elastomeric layer.

6. The article according to claim 1, wherein said outer elastomeric layer is harder than said inner elastomeric layer.

7. The article according to claim 2, wherein said inner elastomeric layer is attached to an annular ridge protruding from the base element.
